(19) United States
(12) Patent Application Publication
(10) Pub. No.: US 2025/0356494 A1
(43) Pub. Date: Nov. 20, 2025

(54) IMAGE PROCESSING DEVICE, ENDOSCOPE, IMAGE PROCESSING METHOD, AND PROGRAM

(71) Applicant: FUJIFILM Corporation, Tokyo (JP)

(72) Inventor: Masaaki OOSAKE, Kanagawa (JP)

(73) Assignee: FUJIFILM Corporation, Tokyo (JP)

(21) Appl. No.: 19/286,356

(22) Filed: Jul. 31, 2025

Related U.S. Application Data
(63) Continuation of application No. PCT/JP2024/002652, filed on Jan. 29, 2024.

(30) Foreign Application Priority Data
Feb. 7, 2023 (JP) 2023-017161

(51) Int. Cl. G06T 7/00; G06T 7/11; G06T 7/13; G06T 7/73

(52) U.S. Cl. CPC G06T 7/0016; G06T 7/11; G06T 7/13; G06T 7/74; G06T 7/75; G06T 2207/10016; G06T 2207/10068

(57) ABSTRACT

An image processing device includes a processor. The processor is configured to: recognize, based on a medical image in which an observation target region is shown, a position of the observation target region in the medical image; determine whether or not to perform output of a size of the observation target region based on the position; and measure the size based on the medical image in a case in which it is determined to perform the output.

### CROSS-REFERENCE TO RELATED APPLICATIONS

This application is a continuation application of International Application No. PCT/JP2024/002652, filed Jan. 29, 2024, the disclosure of which is incorporated herein by reference in its entirety. Further, this application claims priority from Japanese Patent Application No. 2023-017161, filed Feb. 7, 2023, the disclosure of which is incorporated herein by reference in its entirety.

### BACKGROUND

### 1. Technical Field

The technology of the present disclosure relates to an image processing device, an endoscope, an image processing method, and a program.

### 2. Related Art

JP2022-535873A discloses a method of processing a colon image and a video. The method disclosed in JP2022-535873A is a method of generating an instruction to present a graphical user interface (GUI) for dynamically tracking at least one polyp in a plurality of endoscopic images of a colon of a patient.

The method described in JP2022-535873A includes tracking a position of a region in which a polyp is drawn. In addition, the method disclosed in JP2022-535873A includes repeating, for the plurality of endoscopic images, calculating a vector from each endoscopic image to a position of an outer region of each endoscopic image in a case in which the position of the region is outside each endoscopic image, augmenting each endoscopic image by displaying the vector, to generate an augmented endoscopic image, and generating an instruction to display the augmented endoscopic image in the GUI.

JP2020-093076A discloses a medical image processing device comprising an acquisition unit that acquires a tomographic image of an eye to be examined, and a first processing unit that executes first detection processing of detecting at least one retinal layer among a plurality of retinal layers in the acquired tomographic image using a trained model obtained by training using data indicating at least one retinal layer among the plurality of retinal layers in the tomographic image of the eye to be examined.

WO2020/110214A discloses an endoscope system comprising an image input unit that sequentially inputs a plurality of observation images obtained by imaging a subject with an endoscope, a lesion detection unit that detects a lesion portion that is an observation target of the endoscope from the observation image, an overlooking risk analysis unit that determines a degree of an overlooking risk that is a risk that an operator overlooks the lesion portion, based on the observation image, a notification control unit that controls notification means and a notification method for the detection of the lesion portion, based on the degree of the overlooking risk, and a notification unit that notifies the operator of the detection of the lesion portion based on the control of the notification control unit.

In addition, in the endoscope system disclosed in WO2020/110214A, the overlooking risk analysis unit comprises a lesion analysis unit that analyzes the overlooking risk based on a state of the lesion portion. In addition, the lesion analysis unit comprises a lesion size analysis unit that estimates a size of the lesion portion itself. Further, the lesion analysis unit comprises a lesion position analysis unit that analyzes a position of the lesion portion in the observation image.

### SUMMARY

One embodiment according to the technology of the present disclosure provides an image processing device, an endoscope, an image processing method, and a program that enable a user or the like to accurately ascertain a size of an observation target region shown in a medical image.

A first aspect according to the technology of the present disclosure relates to an image processing device comprising: a processor, in which the processor is configured to: recognize, based on a medical image in which an observation target region is shown, a position of the observation target region in the medical image; determine whether or not to perform output of a size of the observation target region based on the position; and output the size in a case in which it is determined to perform the output.

A second aspect according to the technology of the present disclosure relates to the image processing device according to the first aspect, in which the medical image is a plurality of frames in time series, and the processor is configured to: recognize the position in each of the plurality of frames; and determine whether or not to perform the output using an amount of change in the position between the plurality of frames.

A third aspect according to the technology of the present disclosure relates to the image processing device according to the second aspect, in which the amount of change in the position between the plurality of frames is defined based on a distance of the position between the plurality of frames.

A fourth aspect according to the technology of the present disclosure relates to the image processing device according to the second or third aspect, in which the amount of change in the position between the plurality of frames is defined based on a degree of overlap of the observation target region between the plurality of frames.

A fifth aspect according to the technology of the present disclosure relates to the image processing device according to any one of the first to fourth aspects, in which the processor is configured to measure the size by performing AI-based processing on the medical image.

A sixth aspect according to the technology of the present disclosure relates to the image processing device according to any one of the first to fourth aspects, in which the processor is configured to: derive a distance from an observation position to the observation target region by performing AI-based processing on the medical image; and measure the size based on the distance and the number of pixels in a range to be measured in the observation target region.

A seventh aspect according to the technology of the present disclosure relates to the image processing device according to any one of the first to sixth aspects, in which the processor is configured to: determine to perform the output in a case in which the position is present in a first region in the medical image; and determine not to perform the output in a case in which the position is present in a second region outside the first region in the medical image.

An eighth aspect according to the technology of the present disclosure relates to the image processing device according to any one of the first to sixth aspects, in which the medical image is a plurality of frames in time series, and the processor is configured to: recognize the position in each of the plurality of frames; and determine whether or not to perform the output based on an amount of change in the position between the plurality of frames and whether the position is present in a first region in the medical image or a second region outside the first region in the medical image.

A ninth aspect according to the technology of the present disclosure relates to the image processing device according to any one of the first to eighth aspects, in which the medical image is a plurality of frames in time series, and the processor is configured to: recognize the position in each of the plurality of frames by using an AI-based bounding box method; and determine whether or not to perform the output using an amount of change in a bounding box.

A tenth aspect according to the technology of the present disclosure relates to the image processing device according to any one of the first to eighth aspects, in which the medical image is a plurality of frames in time series, and the processor is configured to: recognize the position in each of the plurality of frames by using an AI-based segmentation method; and determine whether or not to perform measurement using an amount of change in a segmentation region.

An eleventh aspect according to the technology of the present disclosure relates to the image processing device according to any one of the first to tenth aspects, in which the processor is configured to determine whether or not to perform the output based on whether or not the position is an edge portion of the medical image.

A twelfth aspect according to the technology of the present disclosure relates to the image processing device according to any one of the first to seventh aspects, in which the medical image is a plurality of frames in time series, and the processor is configured to determine whether or not to perform the output based on the position in a first frame selected in accordance with a given instruction among the plurality of frames and the position in at least one second frame obtained earlier than the first frame among the plurality of frames.

A thirteenth aspect according to the technology of the present disclosure relates to the image processing device according to any one of the first to twelfth aspects, in which the medical image is a moving image.

A fourteenth aspect according to the technology of the present disclosure relates to the image processing device according to any one of the first to twelfth aspects, in which the processor is configured to output the size in a case in which it is determined to perform the output.

A fifteenth aspect according to the technology of the present disclosure relates to the image processing device according to the fourteenth aspect, in which the output of the size is implemented by displaying the size on a first screen.

A sixteenth aspect according to the technology of the present disclosure relates to the image processing device according to any one of the first to fifteenth aspects, in which the processor is configured to output a past result in which the size is measured, in a case in which it is determined not to perform the output.

A seventeenth aspect according to the technology of the present disclosure relates to the image processing device according to the sixteenth aspect, in which the output of the past result is implemented by displaying the past result on a second screen.

An eighteenth aspect according to the technology of the present disclosure relates to the image processing device according to the seventeenth aspect, in which the processor is configured to: display a current result in which the size is measured on the second screen in a case in which it is determined to perform the output; and display the past result and the current result on the second screen in a distinguishable manner depending on whether it is determined not to perform the output or it is determined to perform the output.

A nineteenth aspect according to the technology of the present disclosure relates to the image processing device according to any one of the first to eighteenth aspects, in which the processor is configured to output non-output specifying information for specifying that the output is not to be performed in a case in which it is determined not to perform the output.

A twentieth aspect according to the technology of the present disclosure relates to the image processing device according to the nineteenth aspect, in which the output of the non-output specifying information is implemented by displaying the non-output specifying information on a third screen.

A twenty-first aspect according to the technology of the present disclosure relates to the image processing device according to any one of the first to twentieth aspects, in which the medical image is an endoscopic image obtained by being captured by an endoscope.

A twenty-second aspect of the technology of the present disclosure relates to the image processing device according to any one of the first to twenty-first aspects, in which the observation target region is a lesion.

A twenty-third aspect according to the technology of the present disclosure relates to an endoscope comprising: the image processing device according to any one of the first to twenty-second aspects; and a module that is inserted into a body including the observation target region and that acquires the medical image by imaging the observation target region.

A twenty-fourth aspect according to the technology of the present disclosure relates to an image processing method comprising: recognizing, based on a medical image in which an observation target region is shown, a position of the observation target region in the medical image; determining whether or not to perform output of a size of the observation target region based on the position; and outputting the size in a case in which it is determined to perform the output.

A twenty-fifth aspect according to the technology of the present disclosure relates to a program causing a computer to execute a process comprising: recognizing, based on a medical image in which an observation target region is shown, a position of the observation target region in the medical image; determining whether or not to perform output of a size of the observation target region based on the position; and outputting the size in a case in which it is determined to perform the output.

### BRIEF DESCRIPTION OF THE DRAWINGS

Exemplary embodiments according to the technique of the present disclosure will be described in detail based on the following figures, wherein:

FIG. 1 is a conceptual diagram showing an example of an aspect in which an endoscope is used;

FIG. 2 is a conceptual diagram showing an example of an overall configuration of the endoscope;

FIG. 3 is a block diagram showing an example of a hardware configuration of an electric system of the endoscope;

FIG. 4 is a block diagram showing an example of a main function of a processor included in the endoscope and an example of information stored in an NVM;

FIG. 5 is a conceptual diagram showing an example of processing contents of a recognition unit and a control unit;

FIG. 6 is a conceptual diagram showing an example of processing contents of the recognition unit and a determination unit;

FIG. 7 is a conceptual diagram showing an example of a processing content of the determination unit in a case in which a lesion is included in an edge portion;

FIG. 8 is a conceptual diagram showing an example of a processing content of a measurement unit;

FIG. 9 is a conceptual diagram showing an example of an aspect in which an endoscopic image is displayed on a first screen, and a size is displayed on a second screen;

FIG. 10 is a conceptual diagram showing an example of an aspect in which the endoscopic image is displayed on the first screen, and non-output specifying information is displayed on the second screen;

FIG. 11 is a flowchart showing an example of a flow of medical support processing;

FIG. 12 is a conceptual diagram showing a first modification example of the processing contents of the recognition unit and the determination unit;

FIG. 13 is a flowchart showing a modification example of the flow of the medical support processing;

FIG. 14 is a conceptual diagram showing an example of an aspect in which the endoscopic image is displayed on the first screen, and a past result is displayed on the second screen; and

FIG. 15 is a conceptual diagram showing a second modification example of the processing contents of the recognition unit and the determination unit.

FIG. 16 is a conceptual diagram showing an example of an output destination of the size.

### DETAILED DESCRIPTION

Hereinafter, an example of embodiments of an image processing device, an endoscope, an image processing method, and a program according to the technology of the present disclosure will be described with reference to accompanying drawings.

First, the terms used in the following description will be described.

CPU is an abbreviation for “central processing unit”. GPU is an abbreviation for “graphics processing unit”. RAM is an abbreviation for “random-access memory”. NVM is an abbreviation for “non-volatile memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. AI is an abbreviation for “artificial intelligence”. BLI is an abbreviation for “blue light imaging”. LCI is an abbreviation for “linked color imaging”. I/F is an abbreviation for “interface”. IoU is an abbreviation for “intersection over union”. FIFO is an abbreviation for “first in, first out”.

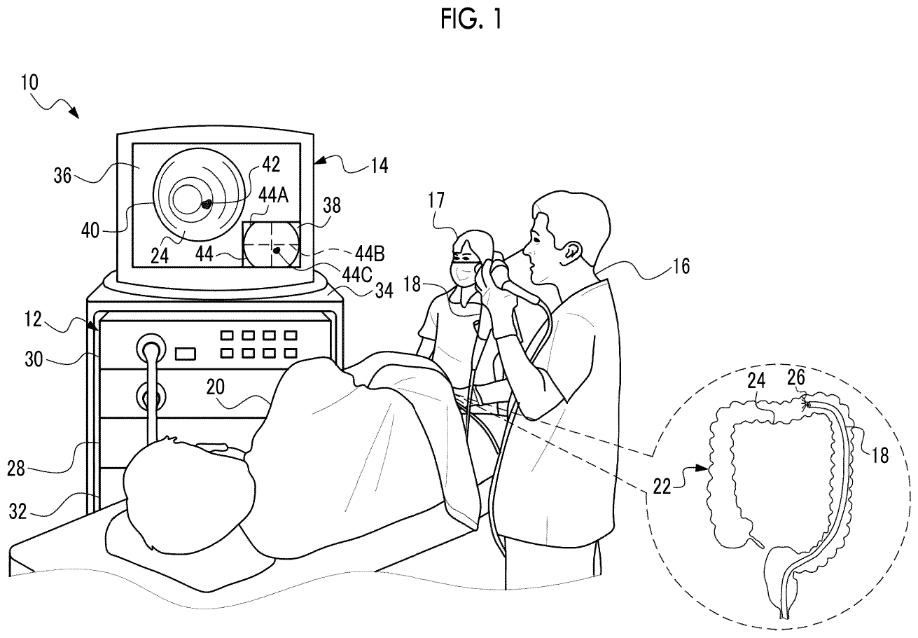

For example, as shown in FIG. 1, an endoscope system 10 comprises an endoscope 12 and a display device 14. The endoscope 12 is used by a doctor 16 in endoscopy. A staff member, such as a nurse 17, assists with the endoscopy. The endoscope 12 in the present embodiment is an example of an “endoscope” according to the technology of the present disclosure.

The endoscope 12 is connected to a communication device (not shown) such that communication can be performed, and information obtained by the endoscope 12 is transmitted to the communication device. Examples of the communication device include a server and/or a client terminal (for example, a personal computer and/or a tablet terminal) that manage various types of information, such as an electronic medical record. The communication device receives information transmitted from the endoscope 12 and executes processing using the received information (for example, processing of storing the information in the electronic medical record or the like).

The endoscope 12 comprises an endoscope body 18. The endoscope 12 is a device for performing medical care for a large intestine 22 included in a body of a subject 20 (for example, a patient) using the endoscope body 18. In the present embodiment, the large intestine 22 is a target that is observed by the doctor 16.

The endoscope body 18 is inserted into the large intestine 22 of the subject 20. The endoscope 12 causes the endoscope body 18 inserted into the large intestine 22 of the subject 20 to image an inside of the large intestine 22 in the body of the subject 20, and performs various medical treatments on the large intestine 22 as necessary.

The endoscope 12 acquires and outputs an image showing an aspect of the inside of the body by imaging the inside of the large intestine 22 of the subject 20. In the present embodiment, the endoscope 12 is an endoscope having an optical imaging function of imaging reflected light obtained by being reflected by an intestinal wall 24 of the large intestine 22 by irradiating the inside of the large intestine 22 with light 26.

It should be noted that, here, the endoscopy of the large intestine 22 has been described as an example, but this is merely an example, and the technology of the present disclosure is applicable to an endoscopy of a luminal organ, such as an esophagus, a stomach, a duodenum, or a trachea.

The endoscope 12 comprises a control device 28, a light source device 30, and an image processing device 32. The control device 28, the light source device 30, and the image processing device 32 are installed in a wagon 34. A plurality of tables are provided in the wagon 34 in an up-down direction, and the image processing device 32, the control device 28, and the light source device 30 are installed from a lower table to an upper table. The display device 14 is installed on an uppermost table in the wagon 34.

The control device 28 controls the entire endoscope 12. The image processing device 32 performs various types of image processing on the image obtained by imaging the intestinal wall 24 with the endoscope body 18, under the control of the control device 28.

The display device 14 displays various types of information including an image. Examples of the display device 14 include a liquid-crystal display and an EL display. A tablet terminal with a display may be used instead of the display device 14 or together with the display device 14.

A plurality of screens are displayed side by side on the display device 14. In the example shown in FIG. 1, a first screen 36 and a second screen 38 are shown as examples of the plurality of screens.

An endoscopic image 40 is displayed on the first screen 36. The endoscopic image 40 is a circular image. That is, the endoscopic image 40 is an image acquired by imaging the intestinal wall 24 with the endoscope body 18 inside the large intestine 22 of the subject 20. The example shown in FIG. 1 shows an image in which the intestinal wall 24 is shown, as an example of the endoscopic image 40. The intestinal wall 24 shown in the endoscopic image 40 includes a lesion 42, and in the example shown in FIG. 1, the lesion 42, which is an observation target region that is gazed at by the doctor 16, is also shown in the endoscopic image 40. The lesion 42 has various types, and examples of the types of the lesion 42 include a neoplastic polyp and a non-neoplastic polyp.

The endoscopic image 40 in the present embodiment is an example of a “medical image”, a “frame”, and an “endoscopic image” according to the technology of the present disclosure. In addition, the lesion 42 in the present embodiment is an example of an “observation target region” and a “lesion” according to the technology of the present disclosure. It should be noted that, here, although the lesion 42 has been described as an example, the technology of the present disclosure is not limited thereto, and the observation target region may be an organ (for example, a duodenal papilla), a marked region, a treated region (for example, a region in which a trace of removal of a polyp or the like remains), or the like.

A moving image is displayed on the first screen 36. The endoscopic image 40 displayed on the first screen 36 is one frame included in a moving image including a plurality of frames in time series. That is, a plurality of frames of the endoscopic images 40 are displayed on the first screen 36 at a predetermined frame rate (for example, 30 frames/see or 60 frames/sec).

Examples of the moving image displayed on the first screen 36 include a moving image in a live view mode. The live view mode is merely an example, and the moving image may be a moving image, such as a moving image in a post view mode, that is temporarily stored in a memory or the like and then displayed. Each frame included in the recorded moving image stored in the memory or the like may be reproduced and displayed on the first screen 36 as the endoscopic image 40.

The second screen 38 is a rectangular screen smaller than the first screen 36. In the example shown in FIG. 1, the second screen 38 is displayed in a superimposed manner on a lower right side of the first screen 36 in front view. Here, the example has been described in which the screen is displayed in a superimposed manner, but this is merely an example, and the screen may be displayed in an embedded manner. In addition, the display position of the second screen 38 may be any position within the screen of the display device 14, but it is preferable that the second screen 38 is displayed at a position at which the comparison with the endoscopic image 40 can be performed. A position specifying image 44 is displayed on the second screen 38. The position specifying image 44 is an image corresponding to the endoscopic image 40, and is an image referred to by a user or the like (for example, the doctor 16) for specifying a position of the lesion 42 in the endoscopic image 40.

The position specifying image 44 has an outer frame 44A, a target mark 44B, and a lesion image 44C. The outer frame 44A is a frame having a shape in which upper and lower portions of an annular frame in which the annular contour of the endoscopic image 40 is reduced are cut out by an upper side and a lower side of the second screen 38.

The target mark 44B is a mark that intersects in a cross shape at the center of a display region of the position specifying image 44. The intersection of the target mark 44B corresponds to a center point of the endoscopic image 40.

The lesion image 44C is an image corresponding to the lesion 42 in the endoscopic image 40, and is displayed in a display aspect in accordance with the size, the shape, and the type of the lesion 42. Examples of the lesion image 44C include a segmentation region itself indicating the lesion 42 recognized by using an AI-based segmentation method for each endoscopic image 40, or an image having a similarity relationship with the segmentation region.

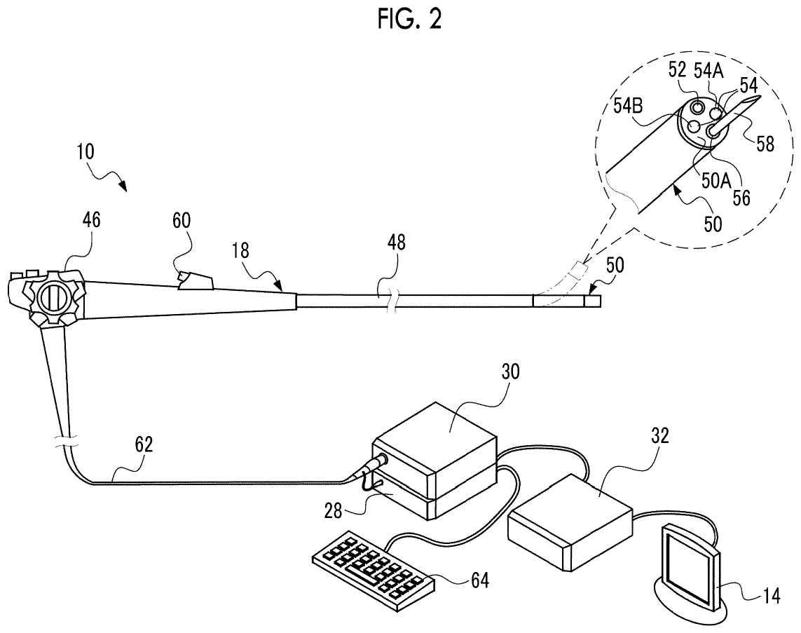

As shown in FIG. 2 as an example, the endoscope body 18 comprises an operating part 46 and an insertion part 48. The insertion part 48 is partially curved by the operation of the operating part 46. The insertion part 48 is inserted into the large intestine 22 while being curved along the shape of the large intestine 22 (see FIG. 1) in accordance with the operation of the operating part 46 by the doctor 16 (see FIG. 1).

A camera 52, an illumination device 54, and a treatment tool opening 56 are provided at a distal end portion 50 of the insertion part 48. The camera 52 and the illumination device 54 are provided on a distal end surface 50A of the distal end portion 50. It should be noted that, here, the form example has been described in which the camera 52 and the illumination device 54 are provided on the distal end surface 50A of the distal end portion 50, but this is merely an example, and the endoscope 12 may be configured as a side-view endoscope by providing the camera 52 and the illumination device 54 on a side surface of the distal end portion 50.

The camera 52 is a device that acquires the endoscopic image 40 as the medical image by imaging the inside of the body (for example, the inside of the large intestine 22) of the subject 20. Examples of the camera 52 include a CMOS camera. However, this is merely an example, and the camera 52 may be other types of cameras, such as a CCD camera. The camera 52 is an example of a “module” according to the technology of the present disclosure.

The illumination device 54 includes illumination windows 54A and 54B. The illumination device 54 emits the light 26 (see FIG. 1) via the illumination windows 54A and 54B. Examples of a type of the light 26 emitted from the illumination device 54 include visible light (for example, white light) and invisible light (for example, near-infrared light). In addition, the illumination device 54 emits special light via the illumination windows 54A and 54B. Examples of the special light include light for BLI and/or light for LCI. The camera 52 images the inside of the large intestine 22 by using an optical method in a state in which the illumination device 54 irradiates the inside of the large intestine 22 with the light 26.

The treatment tool opening 56 is an opening through which a treatment tool 58 protrudes from the distal end portion 50. Further, the treatment tool opening 56 is also used as a suction port for suctioning blood, internal contaminants, and the like and a sending-out port for sending out fluid.

A treatment tool insertion port 60 is formed at the operating part 46, and the treatment tool 58 is inserted into the insertion part 48 through the treatment tool insertion port 60. The treatment tool 58 passes through the insertion part 48 to protrude from the treatment tool opening 56 to the outside. In the example shown in FIG. 2, an aspect is shown in which a biopsy needle protrudes from the treatment tool opening 56 as the treatment tool 58. Here, the biopsy needle has been described as an example of the treatment tool 58, but this is merely an example, and the treatment tool 58 may be grasping forceps, a papillotomy knife, a snare, a catheter, a guide wire, a cannula, and/or a biopsy needle with a guide sheath.

The endoscope body 18 is connected to the control device 28 and the light source device 30 through a universal cord 62. The image processing device 32 and a reception device 64 are connected to the control device 28. In addition, the display device 14 is connected to the image processing device 32. That is, the control device 28 is connected to the display device 14 via the image processing device 32.

It should be noted that, here, since the image processing device 32 has been described as an example of an external device for expanding the functions of the control device 28, the form example has been described in which the control device 28 and the display device 14 are indirectly connected to each other via the image processing device 32, but this is merely an example. For example, the display device 14 may be directly connected to the control device 28. In this case, for example, the functions of the image processing device 32 need only be provided in the control device 28, or the control device 28 need only be provided with a function of directing a server (not shown) to execute the same processing as the processing (for example, medical support processing which will be described below) executed by the image processing device 32, receiving a result of the processing by the server, and using the result.

The reception device 64 receives an instruction from the doctor 16, and outputs the received instruction as an electric signal to the control device 28. Examples of the reception device 64 include a keyboard, a mouse, a touch panel, a foot switch, a microphone, and/or a remote control device.

The control device 28 controls the light source device 30, transmits and receives various signals to and from the camera 52, or transmits and receives various signals to and from the image processing device 32.

The light source device 30 emits light to supply the light to the illumination device 54 under the control of the control device 28. A light guide is provided in the illumination device 54, and the light supplied from the light source device 30 is emitted from the illumination windows 54A and 54B through the light guide. The control device 28 causes the camera 52 to perform imaging, acquires the endoscopic image 40 (see FIG. 1) from the camera 52, and outputs the endoscopic image 40 to a predetermined output destination (for example, the image processing device 32).

The image processing device 32 performs various types of image processing on the endoscopic image 40 input from the control device 28. The image processing device 32 outputs the endoscopic image 40 on which various types of image processing have been performed, to a predetermined output destination (for example, the display device 14).

Here, the form example has been described in which the endoscopic image 40 output from the control device 28 is output to the display device 14 via the image processing device 32, but this is merely an example. For example, an aspect may be adopted in which the control device 28 and the display device 14 are connected to each other, and the endoscopic image 40 on which the image processing has been performed by the image processing device 32 is displayed on the display device 14 via the control device 28.

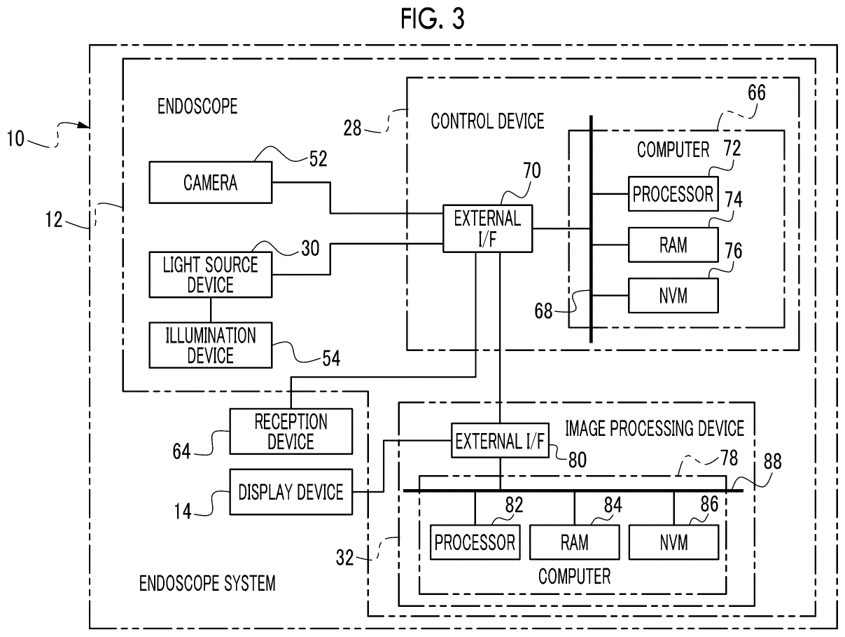

As shown in FIG. 3 as an example, the control device 28 comprises a computer 66, a bus 68, and an external I/F 70. The computer 66 comprises a processor 72, a RAM 74, and an NVM 76. The processor 72, the RAM 74, the NVM 76, and the external I/F 70 are connected to the bus 68.

The processor 72 includes, for example, at least one CPU and at least one GPU, and controls the entire control device 28. The GPU operates under the control of the CPU and is responsible for, for example, the execution of various types of processing of a graphics system and performing calculation using a neural network. It should be noted that the processor 72 may be one or more CPUs integrated with a GPU function, or may be one or more CPUs not integrated with the GPU function. Further, the example shown in FIG. 3 shows an aspect in which one processor 72 is mounted in the computer 66, but this is merely an example, and a plurality of processors 72 may be mounted in the computer 66.

The RAM 74 is a memory that temporarily stores information, and is used as a working memory by the processor 72. The NVM 76 is a non-volatile storage device that stores various programs, various parameters, and the like. Examples of the NVM 76 include a flash memory (for example, an EEPROM and/or an SSD). It should be noted that the flash memory is merely an example, and another non-volatile storage device such as an HDD or a combination of two or more types of non-volatile storage devices may be used.

The external I/F 70 transmits and receives various types of information between one or more devices (hereinafter, also referred to as “first external devices”) existing outside the control device 28 and the processor 72. Examples of the external I/F 70 include a USB interface.

The camera 52 is connected to the external I/F 70 as one of the first external devices, and the external I/F 70 transmits and receives various types of information between the camera 52 and the processor 72. The processor 72 controls the camera 52 through the external I/F 70. In addition, the processor 72 acquires, via the external I/F 70, the endoscopic image 40 (see FIG. 1) obtained by imaging the inside of the large intestine 22 (see FIG. 1) via the camera 52.

The light source device 30 is connected to the external I/F 70 as one of the first external devices, and the external I/F 70 transmits and receives various types of information between the light source device 30 and the processor 72. The light source device 30 supplies light to the illumination device 54 under the control of the processor 72. The illumination device 54 emits the light supplied from the light source device 30.

The reception device 64 is connected to the external I/F 70 as one of the first external devices, and the processor 72 acquires the instruction received by the reception device 64 via the external I/F 70 and executes processing corresponding to the acquired instruction.

The image processing device 32 comprises a computer 78 and an external I/F 80. The computer 78 comprises a processor 82, a RAM 84, and an NVM 86. The processor 82, the RAM 84, the NVM 86, and the external I/F 80 are connected to a bus 88. In the present embodiment, the image processing device 32 is an example of an “image processing device” according to the technology of the present disclosure, the computer 78 is an example of a “computer” according to the technology of the present disclosure, and the processor 82 is an example of a “processor” according to the technology of the present disclosure.

It should be noted that, since a hardware configuration of the computer 78 (that is, the processor 82, the RAM 84, and the NVM 86) is essentially the same as a hardware configuration of the computer 66, the description of the hardware configuration of the computer 78 will not be repeated here.

The external I/F 80 transmits and receives various types of information between one or more devices (hereinafter, also referred to as “second external devices”) existing outside the image processing device 32 and the processor 82. Examples of the external I/F 80 include a USB interface.

The control device 28 is connected to the external I/F 80 as one of the second external devices. In the example shown in FIG. 3, the external I/F 70 of the control device 28 is connected to the external I/F 80. The external I/F 80 transmits and receives various types of information between the processor 82 of the image processing device 32 and the processor 72 of the control device 28. For example, the processor 82 acquires the endoscopic image 40 (see FIG. 1) from the processor 72 of the control device 28 via the external I/Fs 70 and 80, and executes various types of image processing on the acquired endoscopic image 40.

The display device 14 is connected to the external I/F 80 as one of the second external devices. The processor 82 controls the display device 14 via the external I/F 80 such that various types of information (for example, the endoscopic image 40 on which various types of image processing have been performed) are displayed on the display device 14.

In the endoscopy, the doctor 16 determines whether or not a medical treatment is required for the lesion 42 while checking the endoscopic image 40 via the display device 14, and performs the medical treatment on the lesion 42 in a case in which the medical treatment is required. In determining whether or not the medical treatment is required, the size of the lesion 42 is an important determination factor.

In recent years, with the development of machine learning, it has been possible to detect and discriminate the lesion 42 based on the endoscopic image 40 by using an AI method. By applying this technology, it is possible to measure the size of the lesion 42 from the endoscopic image 40.

However, even in a case in which the size of the lesion 42 is measured, there is a concern that the measured size may change significantly depending on an imaging state of the camera 52. For example, in a case in which the camera 52 is moved rapidly or there is a lot of body movement, the endoscopic image 40 is blurred, and thus it is difficult to accurately measure the size of the lesion 42 via the AI-based image processing method. In addition, the edge portion of the endoscopic image 40 is distorted due to the optical influence (for example, aberration) of an objective lens of the camera 52. Therefore, in a case in which the lesion 42 is located at the edge portion of the endoscopic image 40, the lesion 42 is erroneously measured, and the doctor 16 determines whether or not the medical treatment is required in accordance with the erroneously measured size, there is a concern that the medical treatment is performed even though the medical treatment is not actually required, or the medical treatment is not performed even though the medical treatment is actually required.

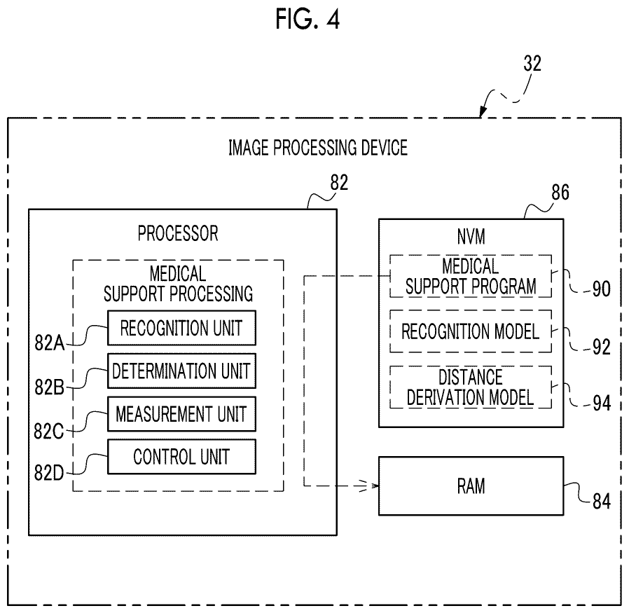

Therefore, in view of such circumstances, in the present embodiment, for example, as shown in FIG. 4, the processor 82 of the image processing device 32 executes the medical support processing.

The NVM 86 stores a medical support program 90. The medical support program 90 is an example of a “program” according to the technology of the present disclosure. The processor 82 reads out the medical support program 90 from the NVM 86 and executes the read out medical support program 90 on the RAM 84 to execute the medical support processing. The medical support processing is implemented by the processor 82 operating as a recognition unit 82A, a determination unit 82B, a measurement unit 82C, and a control unit 82D in accordance with the medical support program 90 executed on the RAM 84.

The NVM 86 stores a recognition model 92 and a distance derivation model 94. The recognition model 92 and the distance derivation model 94 are examples of “AI” according to the technology of the present disclosure. As will be described in detail later, the recognition model 92 is used by the recognition unit 82A, and the distance derivation model 94 is used by the measurement unit 82C.

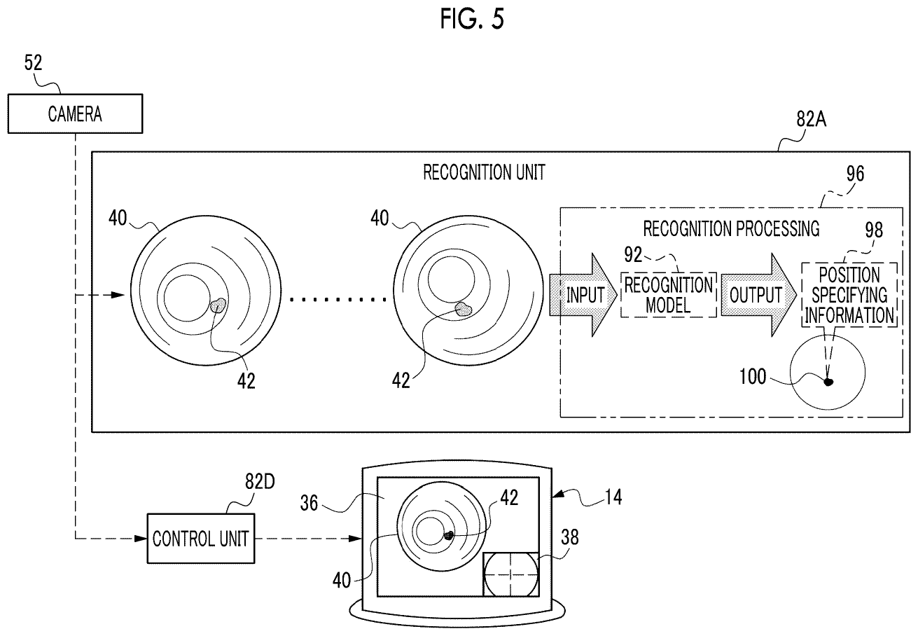

As shown in FIG. 5 as an example, the recognition unit 82A and the control unit 82D acquire the endoscopic image 40 generated by being captured in accordance with an imaging frame rate (for example, several tens of frames/second) by the camera 52 in units of one frame from the camera 52.

The control unit 82D displays the endoscopic image 40 on the first screen 36 as a live view image. That is, each time the control unit 82D acquires the endoscopic image 40 from the camera 52 in units of one frame, the control unit 82D displays the acquired endoscopic images 40 on the first screen 36 in order in accordance with a display frame rate (for example, several tens of frames/second).

The recognition unit 82A recognizes the position of the lesion 42 (that is, the position of the lesion 42 shown in the endoscopic image 40) in the endoscopic image 40 by performing recognition processing 96 on the endoscopic image 40 acquired from the camera 52. The recognition processing 96 is performed on the acquired endoscopic image 40 each time the recognition unit 82A acquires the endoscopic image 40.

The recognition processing 96 is image recognition processing in an AI-based segmentation method. Here, as the recognition processing 96, processing using the recognition model 92 is performed.

The recognition model 92 is a trained model for object detection in the AI-based segmentation method, and is optimized by training a neural network through machine learning using first training data. The first training data is a data set including a plurality of data (that is, data for a plurality of frames) in which first example data is associated with first ground truth data.

The first example data is an image corresponding to the endoscopic image 40. The first ground truth data is ground truth data (that is, an annotation) for the first example data. Here, as an example of the first ground truth data, an annotation for specifying the lesion shown in the image used as the first example data is used.

The recognition unit 82A acquires the endoscopic image 40 from the camera 52, and inputs the acquired endoscopic image 40 to the recognition model 92. As a result, the recognition model 92 specifies a position of a segmentation region 100 identified by the segmentation method as the position of the lesion 42 shown in the input endoscopic image 40 each time the endoscopic image 40 is input, and outputs position specifying information 98 for specifying the position of the segmentation region 100. Examples of the position specifying information 98 include coordinates for specifying the segmentation region 100 in the endoscopic image 40.

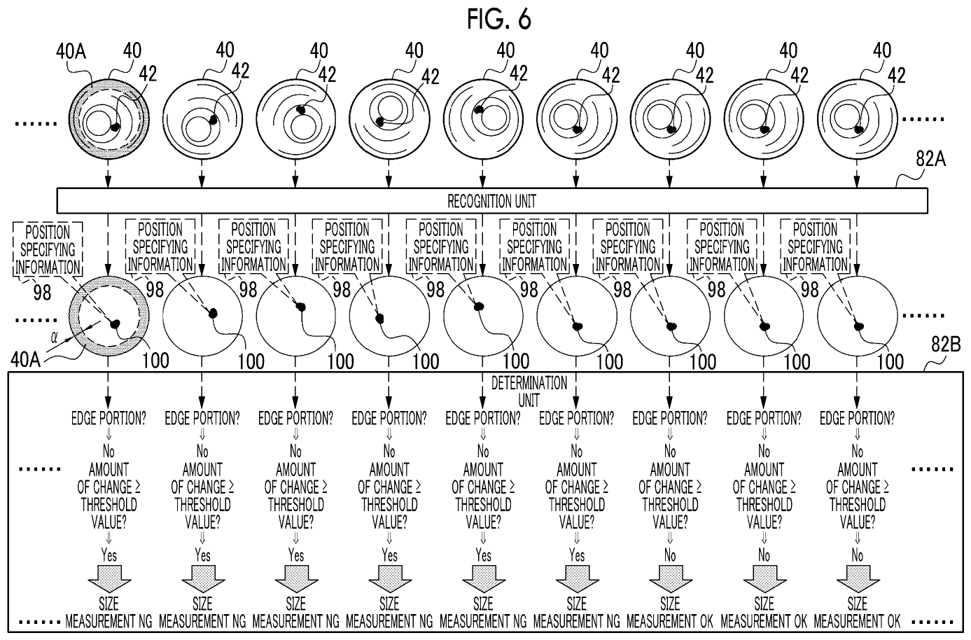

As shown in FIG. 6 as an example, the determination unit 82B acquires the position specifying information 98 from the recognition unit 82A each time the recognition unit 82A performs the recognition processing 96 (see FIG. 5) in units of the endoscopic image 40. Then, the determination unit 82B determines whether or not to perform the output of the size of the lesion 42 based on the position specifying information 98. In the present embodiment, since the size of the lesion 42 is output in a case in which the size of the lesion 42 is measured, and the size of the lesion 42 is not output in a case in which the size of the lesion 42 is not measured, in the example shown in FIG. 6, the determination unit 82B determines whether or not to perform the output of the size of the lesion 42 by determining whether or not to perform the measurement of the size of the lesion 42. That is, performing the measurement of the size of the lesion 42 means performing the output of the size of the lesion 42, and not performing the measurement of the size of the lesion 42 means not performing the output of the size of the lesion 42.

The determination unit 92B determines whether the position of the lesion 42 is at an edge portion 40A of the endoscopic image 40 or at a region other than the edge portion 40A. The edge portion 40A refers to an annular region in which an outer edge of the endoscopic image 40 is an outer periphery, and a circle offset from the outer edge of the endoscopic image 40 to a center side of the endoscopic image 40 by a length a is an inner periphery. The length a may be a fixed value determined in advance as a length for defining the annular region in which the size of the lesion 42 cannot be accurately measured due to the influence of the aberration of the objective lens of the camera 52. Further, the length a may be a variable value that is changed in accordance with an instruction and/or an imaging condition received by the reception device 64 by the user or the like. Here, the region other than the edge portion 40A is an example of a “first region” according to the technology of the present disclosure, and the edge portion 40A is an example of a “second region” and an “edge portion” according to the technology of the present disclosure.

The determination unit 82B determines whether or not the position of the lesion 42 is at the edge portion 40A of the endoscopic image 40 by determining whether or not the entire segmentation region 100 is included in the edge portion 40A based on the position specifying information 98.

Here, in a case in which the entire segmentation region 100 is not included in the edge portion 40A, it is determined that the position of the lesion 42 is not in the edge portion 40A of the endoscopic image 40, and in a case in which the entire segmentation region 100 is included in the edge portion 40A, it is determined that the position of the lesion 42 is at the edge portion 40A of the endoscopic image 40.

It should be noted that, here, whether or not the entire segmentation region 100 is included in the edge portion 40A is used as a determination criterion, but this is merely an example, and whether or not a region of a designated ratio (for example, 80%) in the segmentation region 100 is included in the edge portion 40A may be used as the determination criterion. In addition, the ratio may be a fixed value or a variable value that is changed in accordance with the instruction and/or the imaging condition received by the reception device 64 by the user or the like.

In addition, the determination unit 82B calculates an amount of change in the position of the lesion 42 (hereinafter, also simply referred to as a “lesion position change amount”) between the endoscopic images 40 adjacent in time series. Then, the determination unit 82B determines whether or not the lesion position change amount is equal to or greater than a threshold value. The threshold value may be a fixed value or a variable value that is changed in accordance with the instruction and/or the imaging condition received by the reception device 64 by the user or the like.

In the determination unit 82B, an amount of change in the segmentation region 100 (hereinafter, also referred to as a “segmentation region change amount”) is calculated as the lesion position change amount. Then, it is determined whether or not the segmentation region change amount is equal to or greater than the threshold value. The segmentation region change amount is defined based on a degree of overlap between one segmentation region 100 and the other segmentation region 100 obtained from the endoscopic images 40 adjacent in time series. For example, the segmentation region change amount may be defined based on IoU, or may be simply defined based on the number of pixels in a region in which one segmentation region 100 and the other segmentation region 100 overlap each other.

In a case in which the segmentation region change amount is equal to or greater than the threshold value regardless of whether or not the position of the lesion 42 is at the edge portion 40A, the determination unit 82B determines not to perform the measurement of the size of the lesion 42 (in other words, not to perform the output of the size of the lesion 42). In addition, in a case in which the segmentation region change amount is less than the threshold value for two consecutive frames on the condition that the position of the lesion 42 is not at the edge portion 40A, the determination unit 82B determines to perform the measurement of the size of the lesion 42 (in other words, to perform the output of the size of the lesion 42).

Here, the form example has been described in which the measurement of the size of the lesion 42 is performed on the condition that the segmentation region change amount is less than the threshold value for two consecutive frames, but this is merely an example, and it may be determined to perform the measurement of the size of the lesion 42 on the condition that the segmentation region change amount is less than the threshold value for three or more frames, or it may be determined to perform the measurement of the size of the lesion 42 on the condition that the segmentation region change amount is less than the threshold value for a single frame.

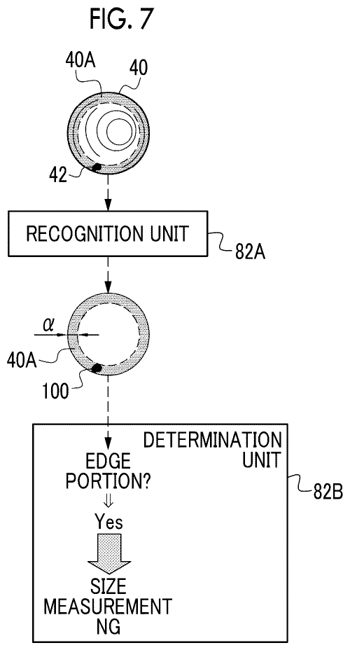

As shown in FIG. 7 as an example, in a case in which the position of the lesion 42 is at the edge portion 40A, the determination unit 82B determines not to perform the measurement of the size of the lesion 42 (in other words, not to perform the output of the size of the lesion 42) regardless of whether or not the lesion position change amount is equal to or greater than the threshold value, and in a case in which the position of the lesion 42 is not at the edge portion 40A, the determination unit 82B determines to perform the measurement of the size of the lesion 42 (in other words, to perform the output of the size of the lesion 42) on the condition that the lesion position change amount is less than the threshold value.

It should be noted that, hereinafter, for convenience of description, the result obtained by determining whether or not to perform the measurement of the size of the lesion 42 via the determination unit 82B is also referred to as a “determination result”.

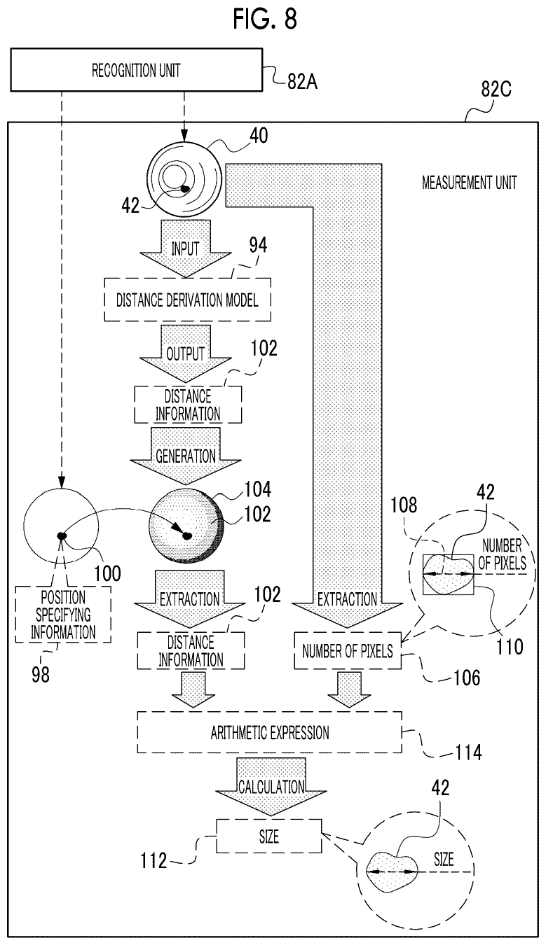

In a case in which the determination unit 82B determines to perform the measurement of the size of the lesion 42, as shown in FIG. 8 as an example, the measurement unit 82C measures a size 112 of the lesion 42 based on the endoscopic image 40. It should be noted that, in a case in which the determination unit 82B determines not to perform the measurement of the size of the lesion 42, the measurement unit 82C does not measure the size 112.

The measurement unit 82C acquires the endoscopic image 40 used for the determination in the determination unit 82B from the recognition unit 82A, and derives distance information 102 based on the acquired endoscopic image 40. The distance information 102 is information indicating a distance from the camera 52 to the intestinal wall 24 (see FIG. 1) including the lesion 42. The distance information 102 is derived for each of all pixels constituting the endoscopic image 40. It should be noted that the distance information 102 may be derived for each block (for example, a pixel group composed of several to several hundred pixels) larger than a pixel in the endoscopic image 40.

The distance information 102 is derived by an AI method. In the present embodiment, the distance derivation model 94 is used to derive the distance information 102.

The distance derivation model 94 is optimized by training a neural network through machine learning using second training data. The second training data is a data set including a plurality of data (that is, data for a plurality of frames) in which second example data is associated with second ground truth data.

The second example data is an image corresponding to the endoscopic image 40. The second ground truth data is ground truth data (that is, an annotation) for the second example data. Here, an annotation for specifying the distance corresponding to each pixel included in the image used as the second example data is used as an example of the second ground truth data.

The measurement unit 82C acquires the endoscopic image 40 used for the determination in the determination unit 82B from the recognition unit 82A and inputs the acquired endoscopic image 40 to the distance derivation model 94. As a result, the distance derivation model 94 outputs the distance information 102 in units of each pixel of the input endoscopic image 40. That is, in the measurement unit 82C, information indicating a distance from a position of the camera 52 (for example, a position of an image sensor or the objective lens mounted in the camera 52) to the intestinal wall 24 shown in the endoscopic image 40 is output from the distance derivation model 94 as the distance information 102 in units of each pixel of the endoscopic image 40. The position of the camera 52 is an example of an “observation position” according to the technology of the present disclosure.

The measurement unit 82C generates a distance image 104 based on the distance information 102 output from the distance derivation model 94. The distance image 104 is an image in which the distance information 102 is distributed in units of pixels included in the endoscopic image 40.

The measurement unit 82C extracts the distance information 102 corresponding to the position specified from the position specifying information 98 from the distance image 104 with reference to the position specifying information 98 obtained based on the endoscopic image 40 input to the distance derivation model 94. Examples of the distance information 102 extracted from the distance image 104 include the distance information 102 corresponding to a specific position (for example, a centroid) of the lesion 42 or a statistic value (for example, a median value, an average value, or a most frequent value) of the distance information 102 for a plurality of pixels (for example, all pixels) included in the lesion 42.

The measurement unit 82C extracts the number of pixels 106 from the endoscopic image 40. The number of pixels 106 is the number of pixels on a line segment 108 in an image region (that is, an image region showing the lesion 42) at the position specified from the position specifying information 98 in the entire image region of the endoscopic image 40 input to the distance derivation model 94. Examples of the line segment 108 include a longest line segment parallel to a long side of a circumscribing rectangular frame 110 in the image region showing the lesion 42. It should be noted that the line segment 108 is merely an example, and a longest line segment parallel to a short side of the circumscribing rectangular frame 110 in the image region indicating the lesion 42 may be applied instead of the line segment 108. The number of pixels 106 in the present embodiment is an example of the “number of pixels” according to the technology of the present disclosure. In addition, the line segment 108 in the present embodiment is an example of a “range to be measured in the observation target region” according to the technology of the present disclosure.

The measurement unit 82C calculates the size 112 of the lesion 42 in the real space based on the distance information 102 extracted from the distance image 104 and the number of pixels 106 extracted from the endoscopic image 40. The size 112 refers to, for example, a length of the lesion 42 in the real space.

An arithmetic expression 114 is used to calculate the size 112. The measurement unit 82C inputs the distance information 102 extracted from the distance image 104 and the number of pixels 106 extracted from the endoscopic image 40, to the arithmetic expression 114. The arithmetic expression 114 is an arithmetic expression in which the distance information 102 and the number of pixels 106 are independent variables and the size 112 is a dependent variable. The arithmetic expression 114 outputs the size 112 corresponding to the input distance information 102 and the input number of pixels 106.

It should be noted that, here, the length of the lesion 42 in the real space has been described as the size 112, but the technology of the present disclosure is not limited thereto, and the size 112 may be a surface area or a volume of the lesion 42 in the real space. In this case, as the arithmetic expression 114, for example, an arithmetic expression is used in which the number of pixels of the entire image region indicating the lesion 42 and the distance information 102 are independent variables and the surface area or volume of the lesion 42 in the real space is a dependent variable.

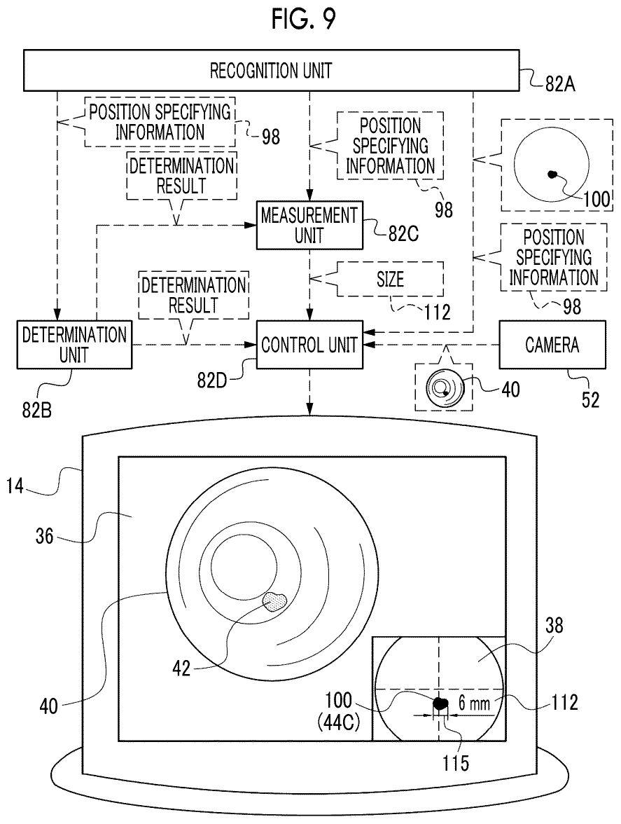

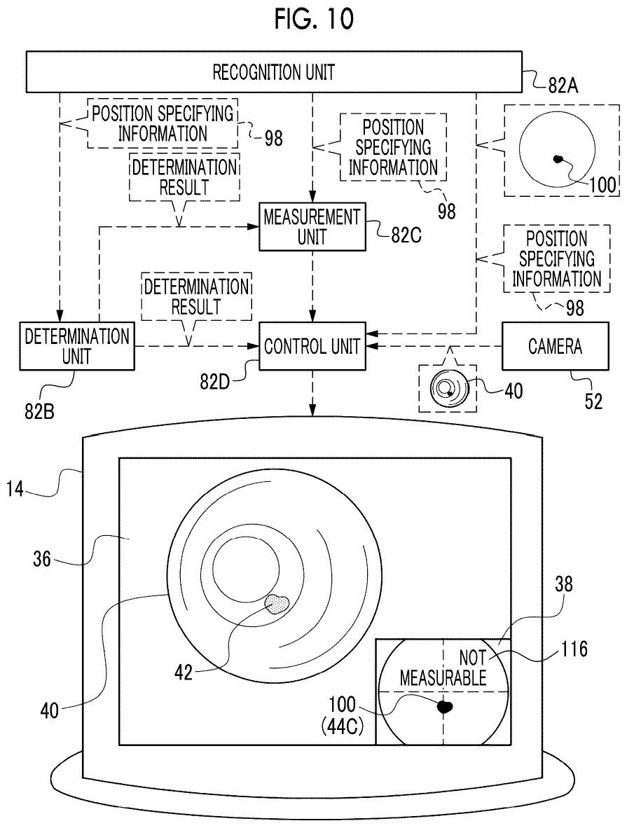

As shown in FIGS. 9 and 10 as an example, the control unit 82D varies a display content to be displayed on the second screen 38 depending on the determination result. As shown in FIG. 9 as an example, in a case in which the determination unit 82B determines to perform the measurement of the size 112, the control unit 82D acquires the endoscopic image 40 used for the determination in the determination unit 82B from the camera 52, and displays the endoscopic image 40 acquired from the camera 52 on the first screen 36.

The control unit 82D acquires the size 112 measured by the measurement unit 82C based on the endoscopic image 40 displayed on the first screen 36 from the measurement unit 82C. In addition, the control unit 82D acquires the segmentation region 100 and the position specifying information 98 corresponding to the endoscopic image 40 displayed on the first screen 36 from the recognition unit 82A.

The control unit 82D displays the segmentation region 100 acquired from the recognition unit 82A on the second screen 38 as the lesion image 44C (see FIG. 1). In this case, the segmentation region 100 is displayed at the position specified from the position specifying information 98 acquired from the recognition unit 82A by the control unit 82D on the second screen 38. In addition, the control unit 82D displays the size 112 acquired from the measurement unit 82C on the second screen 38. In addition, the control unit 82D displays a dimensional line 115 on the second screen 38 such that it is possible to specify which portion of the segmentation region 100 corresponds to the size 112. The dimensional line 115 is created and displayed, for example, by the control unit 82D based on the position specifying information 98 acquired from the recognition unit 82A. The creation of the dimensional line 115 need only be performed in the same manner as the creation of the line segment 108 (that is, in the same manner as the use of the circumscribing rectangular frame 110).

On the other hand, in a case in which the determination unit 82B determines not to perform the measurement of the size 112, as shown in FIG. 10 as an example, the control unit 82D displays the endoscopic image 40 on the first screen 36 and displays the segmentation region 100 on the second screen 38 in the same manner as in the example shown in FIG. 9. In addition, the control unit 82D does not display the size 112 on the second screen 38 but displays non-output specifying information 116 on the second screen 38. The non-output specifying information 116 is information for specifying that the output of the size 112 is not to be performed (in other words, that the measurement of the size 112 is not to be performed) (here, as an example, information for specifying that the determination unit 82B has determined not to perform the measurement of the size 112). In the example shown in FIG. 10, text “not measurable” is displayed on the second screen 38. The text “not measurable” is merely an example, and may be text such as “cannot be output”, and any information may be used as long as the information is information (for example, a mark or a symbol) for specifying that the output of the size 112 is not to be performed.

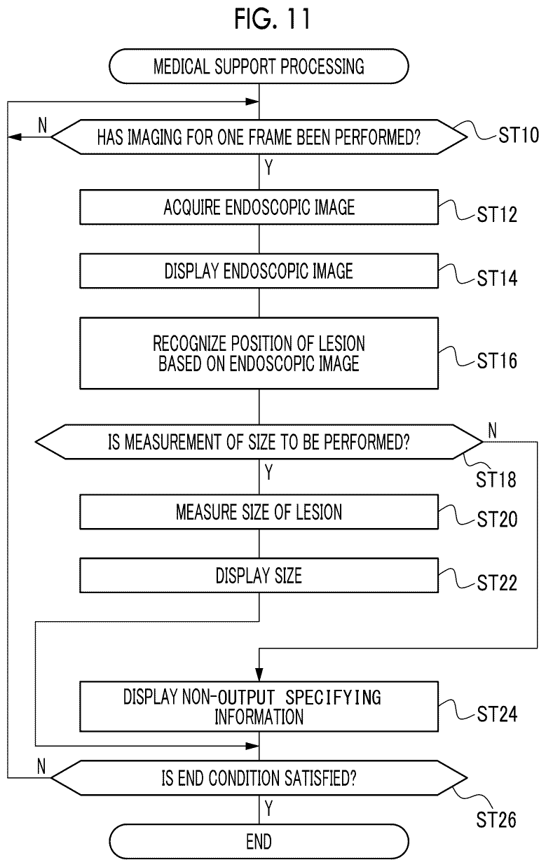

The non-output specifying information 116 in the present embodiment is an example of “non-output specifying information” according to the technology of the present disclosure. In addition, the second screen 38 in the present embodiment is an example of a “first screen”, a “second screen”, and a “third screen” according to the technology of the present disclosure. Hereinafter, the operation of the portion of the endoscope system 10 according to the technology of the present disclosure will be described with reference to FIG. 11.

FIG. 11 shows an example of a flow of the medical support processing performed by the processor 82. The flow of the medical support processing shown in FIG. 11 is an example of an “image processing method” according to the technology of the present disclosure.

In the medical support processing shown in FIG. 11, first, in step ST10, the recognition unit 82A determines whether or not imaging for one frame has been performed by the camera 52 in the large intestine 22. In step ST10, in a case in which the imaging for one frame has not been performed by the camera 52 in the large intestine 22, a negative determination is made, and the determination in step ST10 is performed again. In step ST10, in a case in which the imaging for one frame has been performed by the camera 52 in the large intestine 22, an affirmative determination is made, and the medical support processing proceeds to step ST12.

In step ST12, the recognition unit 82A and the control unit 82D acquire the endoscopic image 40 for one frame obtained by imaging the large intestine 22 with the camera 52 (see FIG. 5). It should be noted that, here, for convenience of description, the description will be made on the premise that the lesion 42 is shown in the endoscopic image 40. After the processing of step ST12 is executed, the medical support processing proceeds to step ST14.

In step ST14, the control unit 82D displays the endoscopic image 40 acquired in step ST12 on the first screen 36 (see FIGS. 5, 9, and 10). After the processing of step ST14 is executed, the medical support processing proceeds to step ST16.

In step ST16, the recognition unit 82A recognizes the position of the lesion 42 in the endoscopic image 40 by performing the recognition processing 96 using the endoscopic image 40 acquired in step ST12, and acquires the position specifying information 98 (see FIG. 5). After the processing of step ST16 is executed, the medical support processing proceeds to step ST18.

In step ST18, the determination unit 82B determines whether or not to perform the measurement of the size 112 of the lesion 42 shown in the endoscopic image 40 acquired in step ST12 based on the position specifying information 98 acquired by the recognition unit 82A in step ST16 (see FIGS. 6 and 7). In step ST18, in a case in which it is determined to perform the measurement of the size 112 of the lesion 42 shown in the endoscopic image 40, an affirmative determination is made, and the medical support processing proceeds to step ST20. In step ST18, in a case in which it is determined not to perform the measurement of the size 112 of the lesion 42 shown in the endoscopic image 40, a negative determination is made, and the medical support processing proceeds to step ST24.

In step ST20, the measurement unit 82C measures the size 112 of the lesion 42 shown in the endoscopic image 40 acquired in step ST12 (see FIG. 8). After the processing of step ST20 is executed, the medical support processing proceeds to step ST22.

In step ST22, the control unit 82D displays the size 112 measured by the measurement unit 82C in step ST20 on the second screen 38 (refer to FIG. 9). After the processing of step ST22 is performed, the medical support processing proceeds to step ST26.

In step ST24, the control unit 82D displays the non-output specifying information 116 on the second screen 38 (see FIG. 10). After the processing of step ST24 is executed, the medical support processing proceeds to step ST26.

In step ST26, the control unit 82D determines whether or not a medical support processing end condition is satisfied. Examples of the medical support processing end condition include a condition that an instruction to end the medical support processing is issued to the endoscope system 10 (for example, a condition that the reception device 64 receives the instruction to end the medical support processing).

In a case in which the medical support processing end condition is not satisfied in step ST26, a negative determination is made, and the medical support processing proceeds to step ST10. In a case in which the medical support processing end condition is satisfied in step ST26, an affirmative determination is made, and the medical support processing ends.

As described above, in the endoscope system 10 according to the present embodiment, the position of the lesion 42 in the endoscopic image 40 is recognized by the recognition unit 82A based on the endoscopic image 40 in which the lesion 42 is shown (see FIG. 5). Here, in a case in which the size 112 of the lesion 42 of which the position in the endoscopic image 40 is recognized by the recognition unit 82A is measured, in a case in which the camera 52 is moved rapidly or there is a lot of body movement, the endoscopic image 40 is blurred, so that it is difficult to accurately measure the size 112 of the lesion 42 by using the AI method using the endoscopic image 40. In addition, in a case in which the edge portion 40A of the endoscopic image 40 is distorted due to the optical influence of the objective lens of the camera 52, it is difficult to accurately measure the size 112 of the lesion 42 via the AI method using the endoscopic image 40. That is, in a case in which the size 112 is measured by a measurement method that does not take into consideration the blurriness of the endoscopic image 40 and/or the distortion of the edge portion 40A (for example, a measurement method in which the distance derivation model 94 created without taking into consideration the blurriness of the endoscopic image 40 and/or the distortion of the edge portion 40A is used), there is a concern that an inaccurate size 112 may be measured.

Therefore, in the endoscope system 10 according to the present embodiment, the determination unit 82B determines whether or not to perform the measurement of the size 112 of the lesion 42 based on the position of the lesion 42 in the endoscopic image 40 used by the recognition unit 82A (see FIGS. 6 and 7). Then, in a case in which the determination unit 82B determines to perform the measurement of the size 112, the measurement unit 82C measures the size 112 of the lesion 42 shown in the endoscopic image 40 based on the endoscopic image 40 used by the recognition unit 82A and the determination unit 82B (see FIG. 8).

Therefore, the doctor 16 can accurately ascertain the size 112 of the lesion 42 shown in the endoscopic image 40. As a result, the doctor 16 can avoid a situation in which the medical treatment is performed even though the medical treatment is not actually required or the medical treatment is not performed even though the medical treatment is actually required.

Further, in the endoscope system 10 according to the present embodiment, the recognition unit 82A recognizes the position of the lesion 42 in the endoscopic image 40 for each endoscopic image 40 (see FIG. 5). Then, it is determined whether or not to perform the measurement of the size 112 of the lesion 42 using the amount of change in the position of the lesion 42 between the endoscopic images 40 adjacent in time series (for example, the segmentation region change amount defined by the IoU) (see FIG. 6). Therefore, even in a case in which the sharpness of the endoscopic image 40 changes or the body movement occurs between the endoscopic images 40 adjacent in time series, the doctor 16 can accurately ascertain the size 112 of the lesion 42 shown in the endoscopic images 40 adjacent in time series.

Further, in the endoscope system 10 according to the present embodiment, the position of the lesion 42 in the endoscopic image 40 is recognized by the recognition unit 82A for each endoscopic image 40 by the AI-based segmentation method (see FIG. 5). Then, the determination unit 82B determines whether or not to perform the measurement of the size 112 of the lesion 42 using the segmentation region change amount (see FIG. 6).

In addition, in the endoscope system 10 according to the present embodiment, whether or not to perform the measurement of the size 112 of the lesion 42 is determined based on whether or not the position of the lesion 42 shown in the endoscopic image 40 is at the edge portion 40A of the endoscopic image 40 (see FIGS. 6 and 7). Therefore, it is possible to prevent the size 112 of the lesion 42 from being inaccurately measured due to the optical influence, such as distortion, on the edge portion 40A of the endoscopic image 40.

In addition, in the endoscope system 10 according to the present embodiment, in a case in which the measurement unit 82C measures the size 112 of the lesion 42, the measured size 112 is displayed on the second screen 38 (see FIG. 9). As a result, the doctor 16 can visually recognize the size 112 of the lesion 42 shown in the endoscopic image 40.

In addition, in the endoscope system 10 according to the present embodiment, in a case in which it is determined not to perform the measurement of the size 112 of the lesion 42, the non-output specifying information 116 is displayed on the second screen 38 (see FIG. 10). The non-output specifying information 116 is information for specifying that the measurement of the size 112 is not to be performed. Therefore, the doctor 16 can visually ascertain that the size 112 of the lesion 42 is not to be measured.

It should be noted that, in the above-described embodiment, although the form example has been described in which the size 112 is displayed in a case in which the measurement of the size 112 is to be performed, the technology of the present disclosure is not limited thereto. For example, in a case in which the determination unit 82B determines not to perform the output of the size 112, the measurement of the size 112 may be performed, but the display of the size 112 may not be performed, and in a case in which the determination unit 82B determines to perform the output of the size 112, the measurement and the display of the size 112 may be performed in the same manner as in the above-described embodiment.

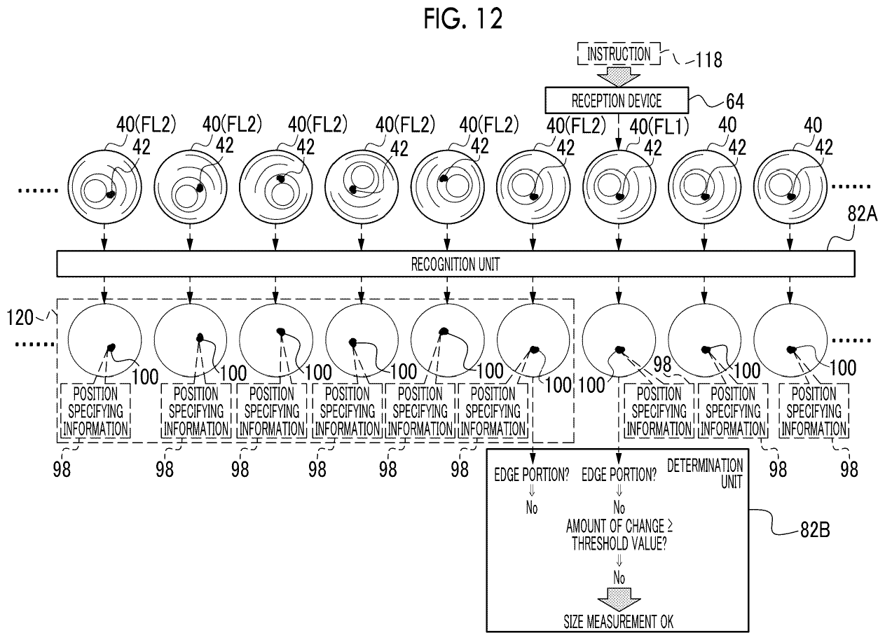

In the above-described embodiment, the determination in the determination unit 82B is performed each time the position specifying information 98 is obtained by the recognition unit 82A, but the technology of the present disclosure is not limited thereto. For example, as shown in FIG. 12, the determination unit 82B may determine whether or not to perform the measurement of the size 112 of the lesion 42 based on the position of the lesion 42 in the endoscopic image 40 (hereinafter, referred to as a “first frame FL1”) selected in accordance with a given instruction (in the example shown in FIG. 12, an instruction 118 received by the reception device 64) among a plurality of endoscopic images 40 in time series and the position of the lesion 42 in at least one endoscopic image 40 (hereinafter, referred to as a “second frame FL2”) obtained earlier than the first frame FL1 among the plurality of endoscopic images 40 in time series. Here, the first frame FL1 is an example of a “first frame” according to the technology of the present disclosure, and the second frame FL2 is an example of a “second frame” according to the technology of the present disclosure.

In the example shown in FIG. 12, an aspect is shown in which a plurality of pieces of the position specifying information 98 corresponding to a plurality of second frames FL2 obtained earlier than the first frame FL1 are stored in a storage region 120. The storage region 120 is, for example, a region provided in the RAM 74. In the storage region 120, a plurality of pieces of the position specifying information 98 corresponding to the second frames FL2 of a specific number of frames (for example, several frames to several hundreds of frames) are stored by a FIFO method. In the example shown in FIG. 12, the determination unit 82B determines whether or not to perform the measurement of the size 112 of the lesion 42 based on the position specifying information 98 selected as a representative and the position specifying information 98 corresponding to the first frame FL1 among the plurality of pieces of position specifying information 98 stored in the storage region 120.

A first example of the position specifying information 98 selected as the representative is, for example, the position specifying information 98 corresponding to the second frame FL2 adjacent to the first frame FL1 in time series. In addition, a second example of the position specifying information 98 selected as the representative is a statistical value (for example, an average value, a median value, or a most frequent value) obtained from the plurality of pieces of position specifying information 98 stored in the storage region 120. In addition, a third example of the position specifying information 98 selected as the representative is the position specifying information 98 randomly selected from the plurality of pieces of position specifying information 98 stored in the storage region 120. In addition, a fourth example of the position specifying information 98 selected as the representative is the position specifying information 98 located at the center in time series among the plurality of pieces of position specifying information 98 stored in the storage region 120. In addition, a fifth example of the position specifying information 98 selected as the representative is the position specifying information 98 selected in accordance with the instruction received by the reception device 64 among the plurality of pieces of position specifying information 98 stored in the storage region 120.

As described above, in the example shown in FIG. 12, the determination of whether or not to perform the measurement of the size 112 of the lesion 42 is made based on the position of the lesion 42 in the first frame FL1 selected in accordance with the instruction 118 received by the reception device 64 among the plurality of endoscopic images 40 in time series and the position of the lesion 42 in at least one second frame FL2 obtained earlier than the first frame FL1 among the plurality of endoscopic images 40 in time series. Therefore, the doctor 16 can determine whether or not to perform the measurement of the size 112 of the lesion 42 shown in the plurality of endoscopic images 40 in time series, at a timing intended by the doctor 16.

In the above-described embodiment, the form example has been described in which the non-output specifying information 116 is displayed on the second screen 38 in a case in which the determination unit 82B determines not to perform the measurement of the size 112 of the lesion 42, but the technology of the present disclosure is not limited thereto. For example, in a case in which the determination unit 82B determines not to perform the measurement of the size 112 of the lesion 42, a past result of the measurement by the measurement unit 82C (that is, the size 112 measured in the past by the measurement unit 82C) may be displayed on the second screen 38.

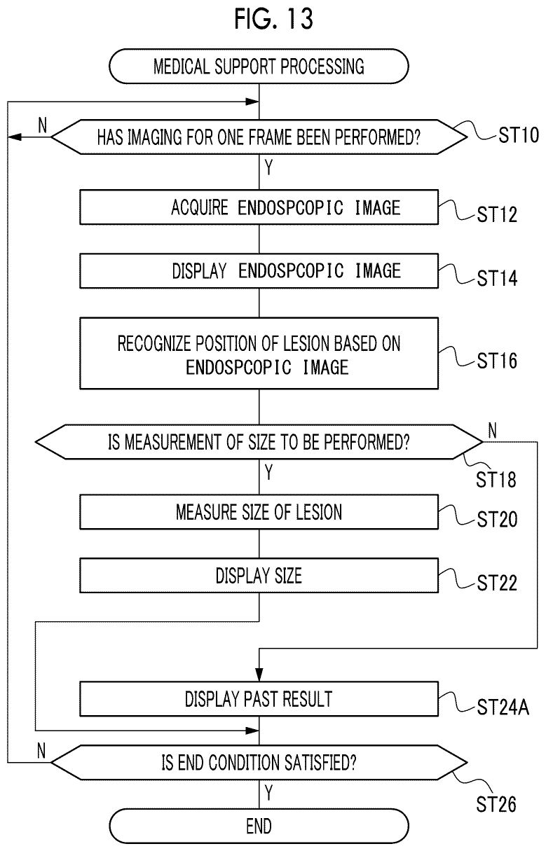

In this case, for example, as shown in FIG. 13, in the medical support processing, the processing of step ST24A is executed instead of the processing of step ST24 shown in FIG. 11.

In step ST24A, the control unit 82D displays the past result of the measurement by the measurement unit 82C (that is, the size 112 measured in the past by the measurement unit 82C) on the second screen 38. A first example of the size 112 measured in the past by the measurement unit 82C is the size 112 previously measured by the measurement unit 82C. A second example of the size 112 measured in the past by the measurement unit 82C is a statistical value (for example, a median value, an average value, a most frequent value, a maximum value, or a minimum value of the size 112 of the lesion 42 shown in several frames to several hundred frames in the past) of the size 112 measured in the past by the measurement unit 82C. A third example of the size 112 measured in the past by the measurement unit 82C is the size 112 measured in a previous endoscopy (for example, the size 112 of the lesion 42 at the same position as the lesion 42 shown in the endoscopic image 40 currently displayed on the first screen 36).

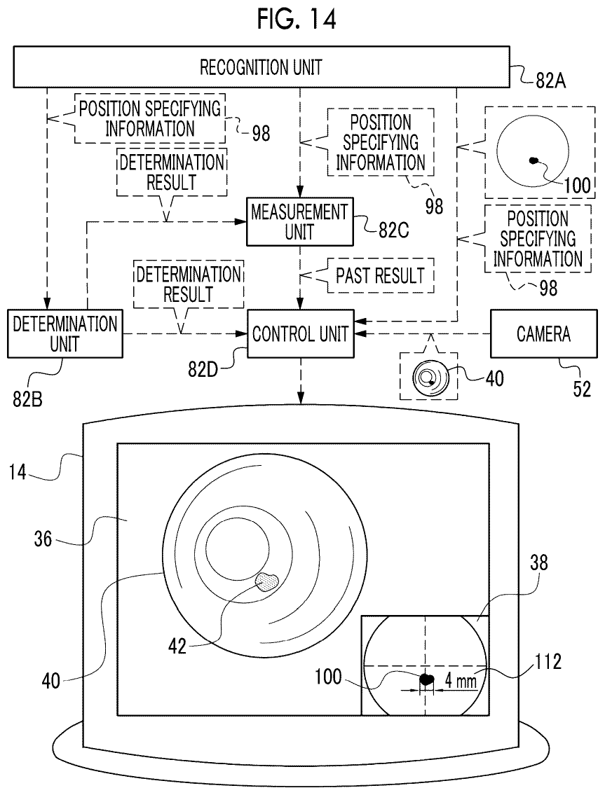

In the example shown in FIG. 14, an aspect is shown in which the size 112 measured in the past is displayed on the second screen 38. In the example shown in FIG. 14, the size 112 displayed on the second screen 38 is displayed in a distinguishable manner from a current result of the measurement by the measurement unit 82C (for example, the size 112 displayed on the second screen 38 shown in FIG. 9). For example, while the size 112 is displayed by a thick line on the second screen 38 shown in FIG. 9, the size 112 is displayed by a thin line on the second screen 38 shown in FIG. 14. The display of the size 112 with the thin line is merely an example, and the size 112 may be displayed in a semi-transparent manner, or may be displayed in a color, a font, or brightness different from the current result of the measurement by the measurement unit 82C, that is, an aspect need only be displayed in which the size 112 displayed on the second screen 38 shown in FIG. 14 is distinguishable from the size 112 displayed on the second screen 38 shown in FIG. 9.

As described above, in the examples shown in FIGS. 13 and 14, in a case in which it is determined not to perform the measurement of the size 112, the past result of the measurement by the measurement unit 82C is displayed on the second screen 38. Therefore, in a case in which it is determined not to perform the measurement of the size 112, the doctor 16 can visually recognize the size 112 measured in the past.

In addition, in the example shown in FIG. 14, the past result of the measurement by the measurement unit 82C (for example, the size 112 displayed on the second screen 38 shown in FIG. 14) is displayed on the second screen 38 in a distinguishable manner from the current result of the measurement by the measurement unit 82C (for example, the size 112 displayed on the second screen 38 shown in FIG. 9). Therefore, the doctor 16 can visually identify the past result of the measurement by the measurement unit 82C and the current result of the measurement by the measurement unit 82C.

In the above-described embodiment, the form example has been described in which the lesion position change amount is calculated based on the degree of overlap of the segmentation regions 100 between the endoscopic images 40 adjacent in time series, but the technology of the present disclosure is not limited thereto. For example, the lesion position change amount may be calculated based on a distance of the position of the lesion 42 between the endoscopic images 40 adjacent in time series. In this case, for example, a distance of a center position of the segmentation region 100 between the endoscopic images 40 adjacent in time series (that is, an amount of deviation of the center position) may be calculated as the lesion position change amount. In this case, the same effect as the effect in the above-described embodiment can be expected.

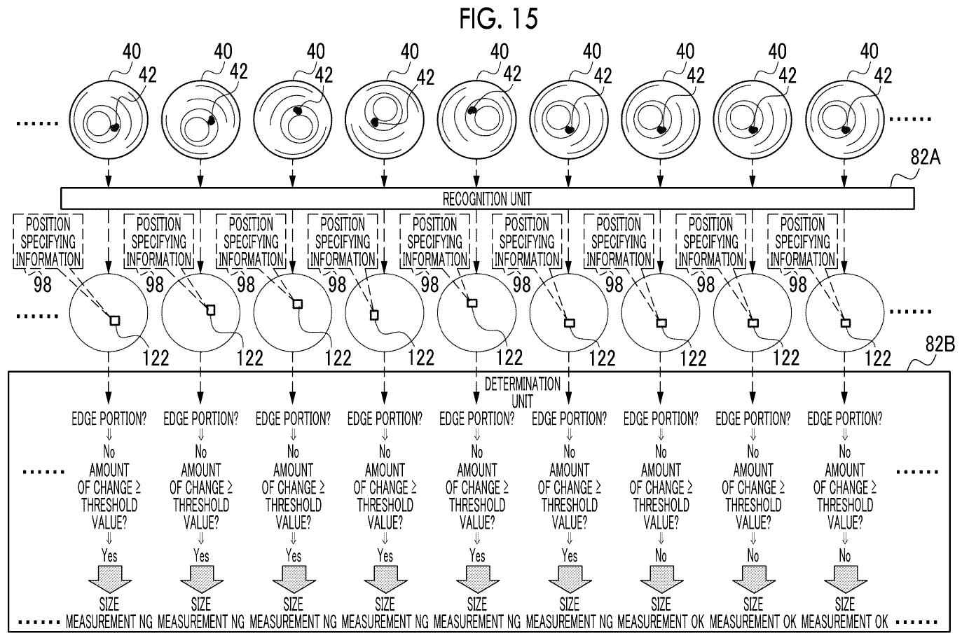

In the above-described embodiment, the form example has been described in which the position of the lesion 42 is recognized by the AI-based segmentation method for each endoscopic image 40, but the technology of the present disclosure is not limited thereto. For example, the position of the lesion 42 may be recognized by an AI-based bounding box method for each endoscopic image 40. In this case, as shown in FIG. 15 as an example, the determination unit 82B need only calculate an amount of change in a bounding box 122, and determine whether or not to perform the measurement of the size 112 of the lesion 42 based on the amount of change in the bounding box 122 in the same manner as in the above-described embodiment. In this case as well, the same effect as the effect in the above-described embodiment can be expected.

In the above-described embodiment, the image recognition processing using the AI method has been described as the recognition processing 96, but the technology of the present disclosure is not limited thereto, and the position of the lesion 42 shown in the endoscopic image 40 may be recognized by the recognition unit 82A by executing image recognition processing using a non-AI method (for example, template matching or the like).

In the above-described embodiment, the form example has been described in which it is determined whether or not to perform the measurement of the size 112 using the amount of change in the position of the lesion 42 between the endoscopic images 40 adjacent in time series, but the technology of the present disclosure is not limited thereto, and it may be determined whether or not to perform the measurement of the size 112 using the amount of change in the position of the lesion 42 between three or more endoscopic images 40 in time series. In this case, the amount of change in the position of the lesion 42 between the three or more endoscopic images 40 in time series may be a statistical value such as an average value, a median value, a mode value, or a maximum value of the amounts of change between the three or more endoscopic images 40 in time series. In addition, it may be determined whether or not the measurement of the size 112 is performed using the amount of change in the position of the lesion 42 between the plurality of frames in time series at intervals of one frame or more.

In the above-described embodiment, the form example has been described in which the size 112 is displayed on the second screen 38, but the technology of the present disclosure is not limited thereto. The size 112 may be, for example, displayed on the first screen 36. In this case, for example, the size 112 may be displayed near the lesion 42 in the endoscopic image 40 or outside the endoscopic image 40. Further, the size 112 may be displayed on a display device different from the display device 14.

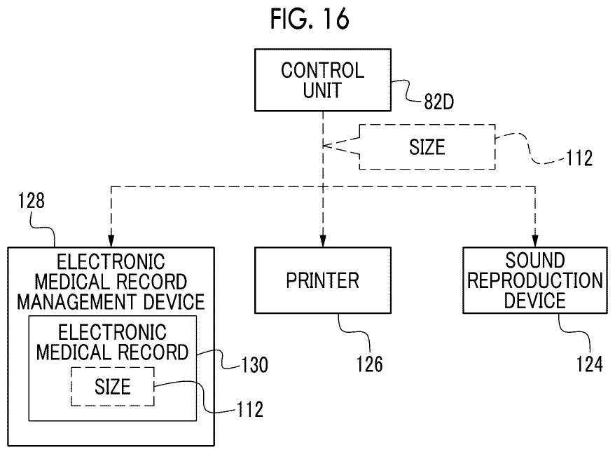

In the above-described embodiment, the display device 14 has been described as the output destination of the size 112, but the technology of the present disclosure is not limited thereto, and the output destination of the size 112 may be other than the display device 14. As shown in FIG. 16 as an example, examples of the output destination of the size 112 include a sound reproduction device 124, a printer 126, and/or an electronic medical record management device 128.

The size 112 may be output as sound by the sound reproduction device 124. Further, the size 112 may be printed on a medium (for example, paper) or the like by the printer 126 as text or the like. In addition, the size 112 may be stored in an electronic medical record 130 managed by the electronic medical record management device 128, along with the display contents of the first screen 36 and/or the second screen 38.

In the above-described embodiment, the form example has been described in which the size 112 is displayed in a case in which the determination unit 82B determines to perform the measurement of the size 112 (in other words, in a case in which the determination unit 82B determines to perform the output of the size 112), and the size 112 is not displayed in a case in which the determination unit 82B determines not to perform the measurement of the size 112 (in other words, in a case in which the determination unit 82B determines not to perform the output of the size 112), but the technology of the present disclosure is not limited thereto. For example, even in a case in which the determination unit 82B determines not to perform the measurement of the size 112, the measurement of the size 112 may be performed, and the measured size 112 may be displayed.

In this case, the concept that the size 112 is not displayed also includes the concept that a display level of the size 112 is lowered. For example, the concept that the size 112 is not displayed also includes the concept that the size 112 is displayed in a display aspect in which the size 112 is not visually perceived by the user or the like (for example, the doctor 16). Examples of the display aspect in this case include display aspects in which a font size of the size 112 is reduced, the size 112 is thinned, the size 112 is dotted, the size 112 is made to blink, the size 112 is displayed for a display time that is imperceptible, and the size 112 is made transparent.

In addition, the concept that the size 112 is not displayed also includes the concept that the size 112 is displayed in a second display aspect different from a first display aspect shown in FIG. 9, although the size 112 is displayed in a display aspect in which the size 112 is visually perceived by the user or the like (for example, the doctor 16). The first display aspect refers to a display aspect (for example, a display aspect defined by a displayed position, a font type, a font size, a font color, and/or brightness) in which it is possible to specify that the determination unit 82B has determined to perform the measurement of the size 112 (in other words, the determination unit 82B has determined to perform the output of the size 112). The second display aspect refers to a display aspect (for example, a display aspect defined by a displayed position, a font type, a font size, a font color, and/or brightness) in which it is possible to specify that the determination unit 82B has determined not to perform the measurement of the size 112 (in other words, the determination unit 82B has determined not to perform the output of the size 112). The first display aspect and the second display aspect are display aspects different from each other, and need only be display aspects in which the determination result can be specified by the user or the like. It should be noted that the same applies to various types of output such as the above-described sound output, printing, and storage.

In the above-described embodiment, the form example has been described in which the non-output specifying information 116 is displayed on the second screen 38, but the form example of output of the non-output specifying information 116 is not limited thereto. For example, the non-output specifying information 116 may be output by the sound reproduction device 124, may be recorded on a medium (for example, paper) by the printer 126, or may be stored in the memory and/or the electronic medical record 130.

In the examples shown in FIGS. 13 and 14, the form example has been described in which the past result of the measurement by the measurement unit 82C is displayed on the second screen 38, but the technology of the present disclosure is not limited thereto. For example, the past result of the measurement by the measurement unit 82C may be output as sound by the sound reproduction device 124, or may be recorded on a medium (for example, paper) by the printer 126.

In the above-described embodiment, the form example has been described in which the arithmetic expression 114 is used to calculate the size 112, but the technology of the present disclosure is not limited thereto, and the size 112 may be measured by performing AI-based processing on the endoscopic image 40. In this case, for example, in a case in which the endoscopic image 40 including the lesion 42 is input, a trained model that outputs the size 112 of the lesion 42 may be used. In a case of creating a trained model, deep learning may be performed on a neural network using training data in which an annotation indicating a size of a lesion is added as ground truth data to a lesion shown in an image used as example data.

In the above-described embodiment, the form example has been described in which the distance information 102 is derived by using the distance derivation model 94, but the technology of the present disclosure is not limited thereto. For example, as another method of deriving the distance information 102 by using the AI method, for example, a method of combining segmentation and depth estimation (for example, regression learning of giving the distance information 102 to the entire image (for example, all pixels constituting the image) or unsupervised learning of learning the distance of the entire image in an unsupervised manner) is used.

In the above-described embodiment, the form example has been described in which the distance from the camera 52 to the intestinal wall 24 is derived by the AI method, but the distance from the camera 52 to the intestinal wall 24 may be actually measured. In this case, for example, a distance measurement sensor may be provided in the distal end portion 50 (see FIG. 2), and the distance from the camera 52 to the intestinal wall 24 may be measured by the distance measurement sensor.

In the above-described embodiment, the endoscopic image 40 has been described as an example, but the technology of the present disclosure is not limited thereto, and the technology of the present disclosure is established even in a case in which a medical image other than the endoscopic image 40 (for example, an image obtained by a modality other than the endoscope 12, such as a radiation image or an ultrasound image) is used.

In the above-described embodiment, the form example has been described in which the size 112 of the lesion 42 shown in the moving image is measured, but this is merely an example, and the technology of the present disclosure is established even in a case in which a frame-by-frame image or a still image in which the lesion 42 is shown is used.

In the above-described embodiment, the form example has been described in which the distance information 102, which is extracted from the distance image 104, is input to the arithmetic expression 114, but the technology of the present disclosure is not limited thereto. For example, the distance information 102 corresponding to the position specified from the position specifying information 98 may be extracted from all pieces of the distance information 102 output from the distance derivation model 94 without generating the distance image 104, and the extracted distance information 102 may be input to the arithmetic expression 114.

In the above-described embodiment, the form example has been described in which the medical support processing is executed by the processor 82 of the computer 78 included in the endoscope 12, but the technology of the present disclosure is not limited thereto, and a device that executes the medical support processing may be provided outside the endoscope 12. Examples of the device provided outside the endoscope 12 include at least one server and/or at least one personal computer that is connected to the endoscope 12 such that the communication can be performed. In addition, the medical support processing may be performed in a distributed manner by a plurality of devices.

In the above-described embodiment, the form example has been described in which the NVM 86 stores the medical support program 90, but the technology of the present disclosure is not limited thereto. For example, the medical support program 90 may be stored in a portable non-transitory computer-readable storage medium such as an SSD or a USB memory. The medical support program 90, which is stored in the non-transitory storage medium, is installed in the computer 78 of the endoscope 12. The processor 82 executes the medical support processing in accordance with the medical support program 90.

The medical support program 90 may be stored in a storage device of another computer or server device connected to the endoscope 12 via a network, and the medical support program 90 may be downloaded in response to a request from the endoscope 12 and installed in the computer 78.

It should be noted that it is not necessary to store the entire medical support program 90 in a storage device of another computer or server device connected to the endoscope 12 or to store the entire medical support program 90 in the NVM 86, and a part of the medical support program 90 may be stored.

The following various processors can be used as hardware resources for executing the medical support processing. An example of the processor is a CPU which is a general-purpose processor that executes software, that is, a program, to function as the hardware resource for executing the medical support processing. In addition, an example of the processor is a dedicated electric circuit which is a processor having a dedicated circuit configuration designed to execute specific processing, such as an FPGA, a PLD, or an ASIC. All processors have a memory built therein or connected thereto, and all processors use the memory to execute the medical support processing.

The hardware resource for executing the medical support processing may be configured by one of the various processors or by a combination of two or more processors of the same type or different types (for example, a combination of a plurality of FPGAs or a combination of a CPU and an FPGA). Further, the hardware resource for executing the medical support processing may be one processor.

A first example of the configuration in which the hardware resource is configured by one processor is an aspect in which one processor is configured by a combination of one or more CPUs and software and functions as the hardware resource for executing the medical support processing. As a second example, as typified by an SoC or the like, there is a form in which a processor that implements all functions of a system including a plurality of hardware resources executing the medical support processing with one IC chip is used. As described above, the medical support processing is implemented by using one or more of the various processors as the hardware resource.

Further, as the hardware structure of the various processors, specifically, an electronic circuit in which circuit elements, such as semiconductor elements, are combined can be used. Further, the above-described medical support processing is merely an example. Therefore, it goes without saying that unnecessary steps may be deleted, new steps may be added, or the processing order may be changed, within a range that does not deviate from the gist of the present disclosure.

The above-described contents and the above-shown contents are detailed descriptions of portions relating to the technology of the present disclosure and are merely examples of the technology of the present disclosure. For example, the description of the configuration, the function, the operation, and the effect above are the description of examples of the configuration, the function, the operation, and the effect of the parts according to the technology of the present disclosure. As a result, it goes without saying that unnecessary parts may be deleted, new elements may be added, or replacements may be made with respect to the above-described contents and the above-shown contents within a range that does not deviate from the gist of the technology of the present disclosure. Further, the description of, for example, common technical knowledge that does not need to be particularly described to enable the implementation of the technology of the present disclosure is omitted in the above-described contents and the above-shown contents in order to avoid confusion and to facilitate understanding of the portions relating to the technology of the present disclosure.

In the present specification, “A and/or B” is synonymous with “at least one of A or B”. That is, “A and/or B” may mean only A, only B, or a combination of A and B. In addition, in the present specification, the same concept as “A and/or B” is applied to a case in which the connection of three or more matters is expressed by “and/or”.

All of the documents, the patent applications, and the technical standards described in the present specification are incorporated into the present specification by reference to the same extent as in a case in which each of the documents, the patent applications, and the technical standards are specifically and individually stated to be described by reference.

In regard to the above-described embodiment, supplementary notes will be further disclosed as follows.

### Supplementary Note 1

An image processing device comprising: a processor, in which the processor is configured to: recognize, based on a medical image in which an observation target region is shown, a position of the observation target region in the medical image; determine whether or not to perform measurement of a size of the observation target region based on the position; and measure the size based on the medical image in a case in which it is determined to perform the measurement.

### Supplementary Note 2

The image processing device according to supplementary note 1, in which the medical image is a plurality of frames in time series, and the processor is configured to: recognize the position for each frame; and determine whether or not to perform the measurement using an amount of change in the position between the plurality of frames.

### Supplementary Note 3

The image processing device according to supplementary note 2, in which the amount of change in the position between the plurality of frames is defined based on a distance of the position between the plurality of frames.

### Supplementary Note 4

The image processing device according to supplementary note 2, in which the amount of change in the position between the plurality of frames is defined based on a degree of overlap of the observation target region between the plurality of frames.

### Supplementary Note 5

The image processing device according to any one of supplementary notes 1 to 4, in which the medical image is a plurality of frames in time series, and the processor is configured to: recognize the position for each frame by using an AI-based bounding box method; and determine whether or not to perform the measurement using an amount of change in a bounding box.

### Supplementary Note 6

The image processing device according to any one of supplementary notes 1 to 4, in which the medical image is a plurality of frames in time series, and the processor is configured to: recognize the position for each frame by using an AI-based segmentation method; and determine whether or not to perform the measurement using an amount of change in a segmentation region.

### Supplementary Note 7

The image processing device according to any one of supplementary notes 1 to 6, in which the processor is configured to determine whether or not to perform the measurement based on whether or not the position is an edge portion of the medical image.

### Supplementary Note 8

The image processing device according to any one of supplementary notes 1 to 7, in which the medical image is a plurality of frames in time series, and the processor is configured to determine whether or not to perform the measurement based on the position in a first frame selected in accordance with a given instruction among the plurality of frames and the position in at least one second frame obtained earlier than the first frame among the plurality of frames.

### Supplementary Note 9

The image processing device according to any one of supplementary notes 1 to 8, in which the medical image is a moving image.

### Supplementary Note 10

The image processing device according to any one of supplementary notes 1 to 9, in which the processor is configured to output the size in a case in which it is determined to perform the measurement.

### Supplementary Note 11

The image processing device according to supplementary note 10, in which the output of the size is implemented by displaying the size on a first screen.

### Supplementary Note 12

The image processing device according to any one of supplementary notes 1 to 11, in which the processor is configured to output a past result of the measurement in a case in which it is determined not to perform the measurement.

### Supplementary Note 13

The image processing device according to supplementary note 12, in which the output of the past result is implemented by displaying the past result on a second screen.

### Supplementary Note 14

The image processing device according to supplementary note 13, in which the processor is configured to: display a current result of the measurement on the second screen in a case in which it is determined to perform the measurement; and display the past result and the current result on the second screen in a distinguishable manner depending on whether it is determined not to perform the measurement or it is determined to perform the measurement.

### Supplementary Note 15

The image processing device according to any one of supplementary notes 1 to 14, in which the processor is configured to output non-measurement specifying information for specifying that the measurement is not to be performed in a case in which it is determined not to perform the measurement.

### Supplementary Note 16

The image processing device according to supplementary note 15, in which the output of the non-measurement specifying information is implemented by displaying the non-measurement specifying information on a third screen.

### Supplementary Note 17

The image processing device according to any one of supplementary notes 1 to 16, in which the medical image is an endoscopic image obtained by being captured by an endoscope.

### Supplementary Note 18

The image processing device according to any one of supplementary notes 1 to 17, in which the observation target region is a lesion.

### Supplementary Note 19

An endoscope comprising: the image processing device according to any one of supplementary notes 1 to 18; and a module that is inserted into a body including the observation target region and that acquires the medical image by imaging the observation target region.

### Supplementary Note 20

An image processing method comprising: recognizing, based on a medical image in which an observation target region is shown, a position of the observation target region in the medical image; determining whether or not to perform measurement of a size of the observation target region based on the position; and measuring the size based on the medical image in a case in which it is determined to perform the measurement.

### Supplementary Note 21

A program causing a computer to execute a process comprising: recognizing, based on a medical image in which an observation target region is shown, a position of the observation target region in the medical image; determining whether or not to perform measurement of a size of the observation target region based on the position; and measuring the size based on the medical image in a case in which it is determined to perform the measurement.

What is claimed is:

1. An image processing device comprising:
a processor,
wherein the processor is configured to:
recognize, based on a medical image in which an observation target region is shown, a position of the observation target region in the medical image;
determine whether or not to perform output of a size of the observation target region based on the position; and
output the size in a case in which it is determined to perform the output.

2. The image processing device according to claim 1,
wherein the medical image is a plurality of frames in time series, and
the processor is configured to:
recognize the position in each of the plurality of frames; and
determine whether or not to perform the output using an amount of change in the position between the plurality of frames.

3. The image processing device according to claim 2,
wherein the amount of change in the position between the plurality of frames is defined based on a distance of the position between the plurality of frames.

4. The image processing device according to claim 2,
wherein the amount of change in the position between the plurality of frames is defined based on a degree of overlap of the observation target region between the plurality of frames.

5. The image processing device according to claim 1,
wherein the processor is configured to measure the size by performing AI-based processing on the medical image.

6. The image processing device according to claim 1,
wherein the processor is configured to:
derive a distance from an observation position to the observation target region by performing AI-based processing on the medical image; and
measure the size based on the distance and the number of pixels in a range to be measured in the observation target region.

7. The image processing device according to claim 1,
wherein the processor is configured to:
determine to perform the output in a case in which the position is present in a first region in the medical image; and
determine not to perform the output in a case in which the position is present in a second region outside the first region in the medical image.

8. The image processing device according to claim 1,
wherein the medical image is a plurality of frames in time series, and
the processor is configured to:
recognize the position in each of the plurality of frames; and
determine whether or not to perform the output based on an amount of change in the position between the plurality of frames and whether the position is present in a first region in the medical image or a second region outside the first region in the medical image.

9. The image processing device according to claim 1,
wherein the medical image is a plurality of frames in time series, and
the processor is configured to:
recognize the position in each of the plurality of frames by using an AI-based bounding box method; and
determine whether or not to perform the output using an amount of change in a bounding box.

10. The image processing device according to claim 1,
wherein the medical image is a plurality of frames in time series, and
the processor is configured to:
recognize the position in each of the plurality of frames by using an AI-based segmentation method; and
determine whether or not to perform the output using an amount of change in a segmentation region.

11. The image processing device according to claim 1,
wherein the processor is configured to determine whether or not to perform the output based on whether or not the position is an edge portion of the medical image.

12. The image processing device according to claim 1,
wherein the medical image is a plurality of frames in time series, and
the processor is configured to determine whether or not to perform the output based on the position in a first frame selected in accordance with a given instruction among the plurality of frames and the position in at least one second frame obtained earlier than the first frame among the plurality of frames.

13. The image processing device according to claim 1,
wherein the processor is configured to output the size in a case in which it is determined to perform the output.

14. The image processing device according to claim 13,
wherein the output of the size is implemented by displaying the size on a first screen.

15. The image processing device according to claim 1,
wherein the processor is configured to output a past result in which the size is measured, in a case in which it is determined not to perform the output.

16. The image processing device according to claim 15,
wherein the output of the past result is implemented by displaying the past result on a second screen.

17. The image processing device according to claim 16,
wherein the processor is configured to:
display a current result in which the size is measured on the second screen in a case in which it is determined to perform the output; and
display the past result and the current result on the second screen in a distinguishable manner depending on whether it is determined not to perform the output or it is determined to perform the output.

18. The image processing device according to claim 1,
wherein the processor is configured to output non-output specifying information for specifying that the output is not to be performed in a case in which it is determined not to perform the output.

19. The image processing device according to claim 18,
wherein the output of the non-output specifying information is implemented by displaying the non-output specifying information on a third screen.

20. The image processing device according to claim 1,
wherein the medical image is an endoscopic image obtained by being captured by an endoscope.

21. The image processing device according to claim 1,
wherein the observation target region is a lesion.

22. An endoscope comprising:
the image processing device according to claim 1; and
a module that is inserted into a body including the observation target region and that acquires the medical image by imaging the observation target region.

23. An image processing method comprising:
recognizing, based on a medical image in which an observation target region is shown, a position of the observation target region in the medical image;
determining whether or not to perform output of a size of the observation target region based on the position; and
outputting the size in a case in which it is determined to perform the output.

24. A non-transitory computer-readable storage medium storing a program executable by a computer to execute a process comprising:
recognizing, based on a medical image in which an observation target region is shown, a position of the observation target region in the medical image;
determining whether or not to perform output of a size of the observation target region based on the position; and
outputting the size in a case in which it is determined to perform the output.
